(19) United States
(12) Patent Application Publication
(10) Pub. No.: US 2013/0259931 A1
(43) Pub. Date: Oct. 3, 2013

(54) ORAL PHARMACEUTICAL COMPOSITIONS OF NEBIVOLOL AND PROCESS FOR THEIR PREPARATION

(71) Applicants: Bandi Parthasaradhi Reddy, Hyderabad (IN); Podili Khadgapathi, Hyderabad (IN); Goli Kamalakar Reddy, Hyderabad (IN)

(72) Inventors: Bandi Parthasaradhi Reddy, Hyderabad (IN); Podili Khadgapathi, Hyderabad (IN); Goli Kamalakar Reddy, Hyderabad (IN)

(21) Appl. No.: 13/436,198

(22) Filed: Mar. 30, 2012

(51) Int. Cl. A61K 31/353; A61K 9/16; A61K 9/28; A61K 9/48; A61P 9/12; A61K 9/14; A61K 9/20

(52) U.S. Cl. 424/452; 424/490; 514/456; 424/465; 427/2.14

(57) ABSTRACT

The present invention is directed to pharmaceutical compositions comprising nebivolol. More particularly, the present invention is directed to oral pharmaceutical compositions comprising nebivolol hydrochloride having a specific surface area of less than 22×10³ cm²/g, and process for preparing the same.

### CROSS-REFERENCE TO RELATED APPLICATION

The application claims the benefit of priority to Indian Application No. 1134/CHE/2011, filed Apr. 1, 2011, under the provisions of 35 U.S.C. §119 and the International Convention for the protection of Industrial Property, which is incorporated herein by reference in its entirety.

### FIELD OF THE INVENTION

The technical field of the present invention relates to pharmaceutical compositions comprising nebivolol or its pharmaceutically acceptable salts and process for preparing the same.

### BACKGROUND OF THE INVENTION

Nebivolol is chemically, (1RS,1′RS)-1,1′[(2RS,2′SR)-bis(6-fluoro-3,4-dihydro-2H-1-benzopyran-2-yl)]-2,2′-iminodiethanol hydrochloride. Nebivolol is a racemate composed of d-Nebivolol and l-Nebivolol with the stereochemical designations of [SRRR]-nebivolol and [RSSS]-nebivolol, respectively. Nebivolol's molecular formula is (C₂₂H₂₅F₂NO₄.HCl) with the following structural formula:

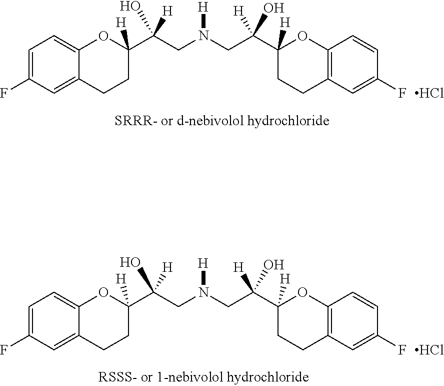

Nebivolol is a beta-adrenergic blocking agent used alone or in combination with other antihypertensive agents for the treatment of Hypertension.

Nebivolol is marketed under the trade name Bystolic® in United States by Forest Labs in the form of oral tablet.

U.S. Pat. No. 4,654,362 describes 2,2′-iminobisethanol derivatives useful for the treatment and/or prevention of disorders of the coronary vascular system.

U.S. Pat. No. 6,545,040 describes [iminobismethylene]bis[6-fluoro-3,4-dihydro-2H-1-benzopyran-2-methanol]derivatives including nebivolol.

U.S. Pat. No. 5,7595,80 assigned to Janssen Pharmaceuticals discloses pharmaceutical compositions of micronized nebivolol having a specific surface area of at least 23×10³ cm²/g (2.3×10³ m²/kg) containing one or more wetting agents.

European Patent 1737847B assigned to Torrent Pharmaceuticals discloses pharmaceutical compositions comprising Nebivolol hydrochloride with out incorporating wetting agent.

European Patent 1886674B assigned to Alfred E Tiefenbacher discloses compositions of micronized nebivolol comprising polyvinylpyrrolidone-co-vinylacetate as carrier matrix polymer.

The existing literature reveals that attempts to use the natural crystalline form of nebivolol have resulted in poor dissolution and bioavailability. Attempts for combining the crystalline form with a wetting agent are also largely unsuccessful. For achieving appropriate dissolution rate or bioavailability, micronized nebivolol is needed.

Micronization of nebivolol hydrochloride requires undue utilities like milling and sifting, which is both expensive and time consuming, and requires the use of wetting agent.

The effect of wetting agents/surfactants over the intestinal membrane is more complex. It has been shown that most wetting agents interact with the absorbing membranes. Permeability enhancement and local damage are closely related sequelae of the interaction of wetting agents with the intestinal wall. Further, the use of wetting agents may facilitate penetration or absorption of endotoxins or pathogenic compounds in to the systemic circulation, which in turn may result in adverse effects on the other.

Thus, there is a need to have compositions of nebivolol free of wetting agents that has better dissolution and bioavailability at the same time being cost and time effective for manufacturing.

The inventors of the present invention have surprisingly found that pharmaceutical compositions comprising non micronized form of nebivolol as the active ingredient, without using wetting agents/surfactants, exhibited excellent dissolution characteristics that were also found to be comparable with respect to the marketed formulation.

### SUMMARY OF THE INVENTION

The present invention provides nebivolol hydrochloride having a specific surface area of less than 22×10³ cm²/g.

The present invention also provides pharmaceutical compositions comprising nebivolol hydrochloride having a specific surface area of less than 22×10³ cm²/g.

The present invention also relates to pharmaceutical composition comprising non micronized form of nebivolol hydrochloride having a specific surface area of less than 22×10³ cm²/g.

In a preferred embodiment, specific surface area of nebivolol ranges from 5×10³ cm²/g to 20×10³ cm²/g.

In an embodiment, the present invention relates to a process for preparing pharmaceutical composition of nebivolol without the use of wetting agent, wherein the process includes granulation/extrusion-spheronization and solution/suspension layering.

In yet another aspect, the present invention provides pharmaceutical composition comprising nebivolol in non micronized form having a specific surface area of less than 22×10³ cm²/g; diluent(s), disintegrant(s), binder(s), lubricant(s) and glidant(s); prepared by granulation/extrusion-spheronization and solution/suspension layering.

In another embodiment, the present invention provides a pharmaceutical composition comprising nebivolol hydrochloride and one or more excipient(s) selected from diluent(s), disintegrant(s), binder(s), lubricant(s) and glidant(s); characterized in that said composition is free of wetting agent and is prepared by granulation/extrusion-spheronization and solution/suspension layering.

In another embodiment, the present invention provides use of methanol in spray granulation process for preparing a dispersion/solution of nebivolol.

In a further aspect, the present invention provides pharmaceutical composition comprising nebivolol, lactose, microcrystalline cellulose, maize starch, pregelatinized starch, crospovidone, croscarmellose sodium, sodium starch glycolate, hydroxy propyl methyl cellulose, colloidal silicon dioxide, magnesium stearate; where in the tablet is prepared by granulation/extrusion-spheronization and solution/suspension layering.

### DETAILED DESCRIPTION OF THE INVENTION

The term “nebivolol” as used here in according to the present invention includes nebivolol in the form of free base, in the form of a pharmaceutically acceptable salt thereof, amorphous nebivolol hydrochloride, crystalline nebivolol hydrochloride or any isomer, derivative, hydrate, solvate, or prodrug or combinations thereof.

The present invention relates to nebivolol hydrochloride having a specific surface area (SSA) of less than 22×10³ cm²/g (2.2×10³ m²/kg).

Pharmaceutical compositions of the present invention comprise non micronized form of nebivolol having a specific surface area of less than 22×10³ cm²/g.

Preferably, according to the present invention comprise nebivolol having a specific surface area in the range from 5×10³ cm²/g to 20×10³ cm²/g.

Specific surface area of nebivolol hydrochloride according to the present invention was measured by B.E.T. (Brunauer-Emmett-Teller) method.

The present invention also provides a process for preparing pharmaceutical compositions of nebivolol by spray/wet granulation/extrusion-spheronization/solution suspension layering.

The present invention also provides a process for preparing pharmaceutical compositions of nebivolol by spray granulation/extrusion-spheronization/wet granulation/solution suspension layering without the use of wetting agent.

Spray granulation/agglomeration process comprise the steps of: (i) preparing a dispersion/solution of nebivolol in a suitable solvent together with one or more excipients followed by, (ii) spray-drying the drug solution of step (i) on one or more excipients to obtain granules, (iii) dry mixing granules obtained in step (ii) with one or more excipients followed by blending, (iv) lubricating and compressing the blend into tablets or filled in to capsules.

Preferred solvent used in spray granulation process for preparing a dispersion/solution of nebivolol is methanol or ethanol.

Extrusion-spheronization process comprise the steps of: (i) blending nebivolol with one or more excipients, (ii) granulating the blended mixture of step no (i) with binder solution to form wet mass, (iii) extruding the wet mass of step no (ii) followed by spheronization using spherodizer to obtain spheroids/spherical granules, and (iv) lubricating the spheroids with all or none or remaining portion of the excipients and compressing in to tablets or filled in to capsules.

Wet granulation process comprise the steps of (i) dry mixing nebivolol with one or more excipients (ii) wet granulating the dry mix of step no (i) using binder solution to form granules followed by drying, (iii) lubricating the dried granules with all or none or remaining portion of the excipients and compressing in to tablets or filled in to capsules.

Solution/Suspension layering comprises, growth of pellets involving deposition of successive layers of solution and/or suspension of drug substance and binders on existing nuclei, which may be inert seed, crystal or granule. The drug particles are dissolved or suspended in the binding liquid, with or without the binder. Droplets of the binding liquid spread on the surface of the nuclei. During drying, liquid evaporates and the dissolved substances crystallize out and capillary forces which are formed draw the particles towards each other and towards the inert seed, forming solid bridges. In suspension layering, particles have low solubility and are bonded by solid bridges formed from the hardening binder i.e., that higher concentration of binder might be necessary.

As a starter seeds usually sugar spheres consisting of a sugar-starch mixture or recently microcrystalline cellulose pellets and the pure drug crystals are used. The most common configuration used is Wurster, bottom spray coater.

Process for preparing a pharmaceutical composition of nebivolol having a specific surface area of less than 22×10³ cm²/g, by solution/suspension layering involves: (i) preparing a dispersion of nebivolol in methanol together with one or more excipients, (ii) coating of the drug dispersion of step (i) on existing nuclei, which may be inert seed, crystal or granule to obtain drug spheres/pellets, (iii) preparation of solution using cushioning agent and coating on to the step (ii) drug pellets, (iv) lubricating the pellets into capsules/compressing the pellets into tablets with extra granular materials.

Pharmaceutical compositions of the present invention include solid dosage forms such as tablets, capsules, granules, MUPS, pellets, solid dispersions, beads, particles, mini-tablets, or orally disintegrating tablets, as well as liquid dosage forms such as solutions, suspensions, syrups, and the like.

Pharmaceutical compositions of nebivolol according to the present invention comprises and one or more diluent(s), binder(s), disintegrant(s), lubricant(s), plasticizer(s), cushioning agent(s) and glidant(s).

Suitable diluents include talc, lactose, sugar, starches, modified starches, mannitol, sorbitol, inorganic salts, cellulose derivatives (e.g. microcrystalline cellulose), calcium sulfate, xylitol, lactitol, starch, pregelatinized starch, kaolin, sucrose, mannitol, sorbitol, dextrates, dextrin, maltodextrin, dextrose, calcium carbonate, dibasic calcium phosphate, tribasic calcium phosphate, magnesium carbonate, magnesium oxide and the like and mixtures thereof.

The term “binders” as used herein is intended to mean substances used to cause adhesion of powder particles in granulation. Suitable binders include, by way of example and without limitation, lactose, starches such as corn starch, potato starch, modified starches, sugars, guar gum, pectin, wax binders, microcrystalline cellulose, methylcellulose, carboxymethylcellulose, hydroxypropyl methylcellulose, hydroxyethyl cellulose, hydroxypropyl cellulose, copolyvidone, sodium alginate, acacia, alginic acid, tragacanth, carboxymethylcellulose sodium, ethyl cellulose, gelatin, liquid glucose, povidone and pregelatinized starch and combination thereof.

The term “disintegrant” as used herein is intended to mean a compound used in solid dosage forms to promote the disruption of the solid mass into smaller particles which are more readily dispersed or dissolved. Suitable disintegrants include, by way of example and without limitation, polacrillin potassium, croscarmellose sodium, crospovidone (e.g., KOLLIDON®, POLYPLASDONE®), polyvinylpyrrolidone, sodium starch glycolate (e.g., PRIMOGEL, EXPLOTAB®), hydroxypropyl methylcellulose, hydroxypropyl cellulose, carboxymethyl cellulose calcium, starches such as corn starch, potato starch, pre-gelatinized and modified starches, clays, bentonite, microcrystalline cellulose (e.g. Avicel™), carsium (e.g. Amberlite™), alginates, gums such as agar, guar, locust bean, karaya, pectin, tragacanth and the like or combinations thereof.

Suitable lubricants include, by way of example and without limitation, calcium stearate, magnesium stearate, sodium stearyl fumarate, zinc stearate, mineral oil, stearic acid, fumaric acid, palmitic acid, talc, carnauba wax, hydrogenated vegetable oils, mineral oil, polyethylene glycols and the like or combinations thereof.

The term “glidant” as used herein is intended to mean agents used in tablet and capsule formulations to improve flow-properties during tablet compression and filling in to capsules to produce an anti-caking effect. Such compounds include, by way of example and without limitation, colloidal silica, calcium silicate, magnesium silicate, silicon hydrogel, cornstarch, talc and the like or combinations thereof.

Suitable cushioning agent(s) include, by way of example and without limitation, microcrystalline cellulose, PEG, waxes, polyvinyl acetate and the like or combinations thereof.

Suitable plasticizers include, by way of example and without limitation, glyceryl monostearate, triethyl citrate, macrogols, lactic acid, lactic acid acetamide, sorbitol, glycerin, triacetin, acetyl triethyl citrate, acetyl tributyl citrate, dibutyl phthalate, polyvinylpyrrolidone, triethylene glycol, tricresyl phosphate, dibutyl tartrate, ethylene glycol monooleate, palmitic acid, stearic acid, oleic acid, dibutyl sebacate, acetylated monoglycerides, cetyl alcohol and other hydrogenated oils and waxes, as well as polyethylene glycol 300, 400, 600, 1450, 3350 and 8000 and the like or combinations thereof.

The tablets of the present invention may be optionally coated with an aqueous or non aqueous solution or dispersion of film forming agents.

The invention is further exemplified with following examples and is not intended to limit the scope of the inventions. It is obvious to those skilled in the art to find out the composition for other dosage forms and substitute the equivalent excipients as described in this specification or with the one known to the industry.

### EXAMPLES 1-2

### Pharmaceutical Compositions of Nebivolol Prepared by Wet Granulation:

| | | | |
|---|---|---|---|
| | | Example-1 | Example-2 |
| S. No | Ingredients | Mg/tablet | Mg/tablet |

Intragranular

| | | | |
|---|---|---|---|
| 1 | Nebivolol HCl having SSA of | 22 | 22 |
| | 14.2 cm²/gm | | |
| 2 | Lactose monohydrate | — | 71 |
| 3 | Sodium starch glycolate | — | 7 |
| 4 | Pregelatinized starch | 35 | 30 |
| 5 | Microcrystalline cellulose | 66 | — |
| 6 | Croscarmellose sodium | 7 | — |

Granulating agent

| | | | |
|---|---|---|---|
| 7 | Purified water | 56 | 70 |
| 8 | Hydroxypropyl methyl cellulose | 4.6 | — |

Extra granular

| | | | |
|---|---|---|---|
| 9 | Microcrystalline cellulose | 86.4 | 91 |
| 10 | Sodium starch glycolate | — | 7 |
| 11 | Croscarmellose sodium | 7 | — |
| 12 | Colloidal silicon dioxide | 0.5 | 0.5 |
| 13 | Magnesium stearate | 1.5 | 1.5 |

| | | |
|---|---|---|
| TOTAL WEIGHT | 230 | 230 |

### Brief Manufacturing Process:

i) intragranular materials were sifted through #30 mesh and blended together,
ii) the blended material of step no (i) was loaded in a rapid mixer granulator and granulated using binder solution,
iii) the granules of step no (ii) were dried and sifted through #30 mesh,
iv) extra granular magnesium stearate was sifted through #40 mesh,
v) extra granular materials were sifted together through #30 mesh,
vi) materials of step (iii), (iv) and (v) were blended together and compressed into tablets or filled in to capsules.

### EXAMPLE 3

### Pharmaceutical Compositions of Nebivolol Prepared by Spray Granulation:

| | | |
|---|---|---|
| S. NO | INGREDIENTS | MG/UNIT |

DRYMIX

| | | |
|---|---|---|
| 1 | Lactose monohydrate | 125 |
| 2 | Maize starch | 40 |
| 3 | Crospovidone | 15 |

DRUG SOLUTION

| | | | |
|---|---|---|---|
| 4 | Nebivolol HCl | 22 | |
| 5 | Hydroxypropyl methyl cellulose | 6 | |
| 6 | Methanol | 0.5 | ml |
| 7 | Purified water | 120 | |

EXTRA GRANULAR

| | | |
|---|---|---|
| 8 | Microcrystalline cellulose | 35.5 |
| 9 | Crospovidone | 5 |
| 10 | Colloidal silicon dioxide | 0.5 |
| 11 | Magnesium stearate | 1 |

| | |
|---|---|
| TOTAL WEIGHT | 250 |

### Brief Manufacturing Process:

i) materials of dry mix were sifted through #30 mesh and mixed together in fluid bed granulator,
ii) nebivolol was dispersed in required quantity of methanol,
iii) hydroxypropyl methyl cellulose was dissolved in required quantity of purified water,
iv) solution of step (iii) was added to the solution of step (ii) under stirring,
v) solution obtained in step (iv) was sprayed on to drymix of step (i) to form granules,
vi) granules of step no (v) were dried,
vii) extra granular magnesium stearate was sifted through #40 mesh,
viii) extra granular materials were sifted together through #30 mesh,
ix) granules of step (vi) and (viii) were sifted through #30 mesh and blended for 10 min,
x) magnesium stearate of step (vii) was blended together with materials of step (ix) and compressed into tablets or filled in to capsules.

### EXAMPLE 4

### Pharmaceutical Compositions of Nebivolol Prepared by Extrusion-Spheronization:

| | | |
|---|---|---|
| S. NO | INGREDIENTS | MG/UNIT |

INTRAGRANULAR

| | | |
|---|---|---|
| 1 | Nebivolol HCl | 22 |
| 2 | Lactose monohydrate | 71 |
| 3 | Microcrystalline cellulose | 30 |
| 4 | Croscarmellose sodium | 7 |

BINDER SOLUTION

| | | |
|---|---|---|
| 5 | Hydroxypropyl methyl cellulose | 12 |
| 6 | Purified water | 30 |

EXTRA GRANULAR

| | | |
|---|---|---|
| 7 | Colloidal silicon dioxide | 0.5 |
| 8 | Magnesium stearate | 0.5 |

| | |
|---|---|
| TOTAL WEIGHT | 143 |

### Brief Manufacturing Process:

i) hydroxypropyl methyl cellulose was dissolved in required quantity of purified water to form binder solution,
ii) intra granular materials were dry mixed and granulated with binder solution of step (i) using rapid mixer granulator,
iii) the wet granules of step no (ii) was extruded and the resulted extrudes were spheronized using spherodizer to obtain spheroids/spherical granules,
iv) spherical granules of step (iii) were dried completely, and
v) granules of step (iv) were lubricated with extra granular materials and compressed or filled in to capsules.

### EXAMPLE 5

### Pharmaceutical Compositions of Nebivolol Prepared by Top Spray Granulation:

| | | |
|---|---|---|
| S. NO | INGREDIENTS | MG/UNIT |

DRYMIX

| | | |
|---|---|---|
| 1 | Lactose monohydrate | 123.00 |
| 2 | Maize starch | 46.00 |
| 3 | Sodium starch glycolate | 13.00 |

DRUG SOLUTION

| | | |
|---|---|---|
| 4 | Nebivolol HCl having SSA of 10.7 cm²/gm | 22.00 |
| 5 | Hydroxypropyl methyl cellulose | 6.00 |
| 6 | Methanol | q.s |
| 7 | Purified water | q.s |

EXTRA GRANULAR

| | | |
|---|---|---|
| 8 | Microcrystalline cellulose | 18.00 |
| 9 | Colloidal silicon dioxide | 0.5 |
| 10 | Magnesium Stearate | 1.5 |

| | |
|---|---|
| TOTAL WEIGHT | 230.00 |

### Brief Manufacturing Process:

i) lactose monohydrate, maize starch and sodium starch glycolate were sifted through #30 mesh and mixed together in a top spray fluid bed granulator,
ii) nebivolol was dispersed/dissolved in required quantity of methanol,
iii) hydroxypropyl methyl cellulose was dissolved in required quantity of purified water,
iv) solution of step (iii) was added to the solution of step (ii) under stirring,
v) solution obtained in step (iv) was sprayed on to drymix of step (i) to form granules and the granules were dried,
vi) extra granular magnesium stearate was sifted through #40 mesh,
vii) extra granular microcrystalline cellulose and colloidal silicon dioxide were sifted separately through #30 mesh,
viii) granules of step (v) and (vii) were sifted through #30 mesh and blended for 10 min,
ix) magnesium stearate of step (vi) was blended together with materials of step (viii) and compressed into tablets or filled in to capsules.

### EXAMPLE 6

### Pharmaceutical Compositions of Nebivolol Prepared by Top Spray Granulation:

| | | |
|---|---|---|
| S. NO | INGREDIENTS | MG/UNIT |

DRYMIX

| | | |
|---|---|---|
| 1 | Microcrystalline cellulose | 90.00 |
| 2 | Maize starch | 60.00 |
| 3 | Copovidone | 30.00 |

DRUG DISPERSION

| | | | |
|---|---|---|---|
| 4 | Nebivolol HCl | 22.00 | |
| 5 | Methanol | 0.25 | ml |
| 6 | Purified water | 120 | |

EXTRA GRANULAR

| | | |
|---|---|---|
| 7 | Microcrystalline cellulose | 26.00 |
| 8 | Colloidal silicon dioxide | 0.5 |
| 9 | Magnesium stearate | 1.5 |

| | |
|---|---|
| TOTAL WEIGHT | 230.00 |

### Brief Manufacturing Process:

i) microcrystalline cellulose, maize starch and copovidone were sifted through #30 mesh and blended together in a top spray fluid bed granulator,
ii) nebivolol was dispersed in required quantity of methanol,
iii) required quantity of purified water was added to step no. (ii) under stirring.
iv) drug solution of step no (ii) was sprayed on to the dry mix of step (i) to form granules and the granules were dried,
v) extra granular magnesium stearate was sifted through #40 mesh,
vi) extra granular microcrystalline cellulose and colloidal silicon dioxide were sifted through #30 mesh separately,
vii) granules of step (iv) and (vi) were sifted through #40 mesh for 10 min,
viii) magnesium stearate of step (v) was blended together with materials of step (vii) and compressed into tablets or filled in to capsules.

### EXAMPLE 7

### Nebivolol Capsules Prepared by Solution & Suspension Layering:

| | | |
|---|---|---|
| S. NO | INGREDIENTS | MG/UNIT |

Drug loading

| | | |
|---|---|---|
| 1 | Sugar spheres | 96.00 |
| 2 | Nebivolol hydrochloride | 22.00 |
| 3 | Hydroxyl propyl cellulose | 10 |
| 5 | Hydroxypropyl cellulose, low- | 4.00 |
| | substituted | |
| 6 | Talc | 1.00 |
| 7 | Methanol | q.s |
| 8 | Water | q.s |

Lubrication

| | | |
|---|---|---|
| 11 | Talc | 1.5 |
| 12 | Colloidal silicon dioxide | 0.5 |

| | |
|---|---|
| Total | 135.00 |

### Brief Manufacturing Process:

i) binder solution was prepared by dissolving hydroxyl propyl cellulose in required quantity of water,
ii) nebivolol hydrochloride was dissolved/dispersed in required quantity of methanol,
iii) the solution of step no. (i) was added to step no. (ii) under stirring,
iv) to the solution/dispersion of step no. (iii), low-substituted hydroxypropyl cellulose was added under stirring.
v) antitacking agent talc was added to the solution/dispersion of step no. (iv) under stirring to get uniform dispersion.
vi) the dispersion of step no. (v) was coated on to the inert sugar spheres,
vii) the coated beads/spheres were lubricated with talc and colloidal silicon dioxide and filled into capsules.

### EXAMPLE 8

### Pharmaceutical Compositions of Nebivolol Prepared by Solution & Suspension Layering:

| | | |
|---|---|---|
| S. NO | INGREDIENTS | MG/UNIT |

Drug loading

| | | |
|---|---|---|
| 1 | Celpheres | 97.00 |
| 2 | Nebivolol hydrochloride | 22.00 |
| 3 | Sodium CMC | 9.00 |
| 4 | Sodium starch glycolate | 4.00 |
| 5 | Talc | 1.00 |
| 6 | Methanol | q.s |
| 7 | Water | q.s |

Cushioning layer

| | | |
|---|---|---|
| 8 | Glyceryl monostearate | 0.75 |
| 9 | Talc | 0.25 |
| 10 | Isopropyl alcohol | q.s |

Extrgranular materials

| | | |
|---|---|---|
| 11 | Microcrystalline cellulose | 90.0 |
| 12 | Sodium starch glycolate | 4.00 |
| 13 | Colloidal silicon dioxide | 0.5 |
| 14 | Magnesium stearate | 1.5 |

| | |
|---|---|
| Total | 230.00 |

### Brief Manufacturing Process:

i) sodium CMC was dissolved in required quantity of water,
ii) nebivolol hydrochloride was dissolved/dispersed in required quantity of methanol,
iii) the solution of step no. (i) was added to step no. (ii) under stirring,
iv) to the solution/dispersion of step no. (iii), disintegrant sodium starch glycolate was added under stirring,
v) antitacking agent talc was added to the solution/dispersion of step no. (iv) under stirring to get uniform dispersion,
vi) the dispersion of step no. (v) was coated on to the inert Celpheres to obtain drug loaded spheres,
vii) glyceryl monostearate was dissolved in required quantity of isopropyl alcohol,
viii) talc was added to the solution of step no. (vii) under stirring to get uniform dispersion,
ix) the dispersion of step no. (viii) was coated on to the obtained drug loaded spheres of step no. (vi) to obtained cushioned pellets,
x) extrgranular materials were sifted through #40 mesh,
xi) the cushioned beads/pellets of step no. (ix) and extrgranular materials of step no. (x) were sifted together through #20 mesh and compressed to tablets.

1. A pharmaceutical composition comprising nebivolol hydrochloride, having a specific surface area of less than 22×10³ cm²/g, wherein the composition comprises an inert seed, crystal or granule having deposited thereon the nebivolol hydrochloride and at least one pharmaceutically acceptable excipient.

2. The pharmaceutical composition of claim 1, wherein the at least one pharmaceutically acceptable excipient is selected from a diluent, a lubricant, a binder, a plasticizer, cushioning agent and a disintegrant, and where-in said pharmaceutical composition is free of wetting agent.

3. (canceled)

4. (canceled)

5. (canceled)

6. (canceled)

7. (canceled)

8. (canceled)

9. (canceled)

10. The pharmaceutical composition of claim 1 in the form of tablets, capsules, granules, pellets, solid dispersions, beads, particles, or mini-tablets.

11. A process for preparing a pharmaceutical composition of nebivolol having a specific surface area of less than 22×10³ cm²/g, by solution/suspension layering comprising:
(i) preparing a dispersion of nebivolol in methanol together with one or more excipients,
(ii) coating the drug dispersion of step (i) on an inert seed, crystal or granule to provide drug pellets,
(iii) preparing a solution comprising a cushioning agent and coating the solution onto the step (ii) drug pellets to form coated drug pellets, and
(iv) lubricating the coated drug pellets and loading the coated drug pellets into capsules or compressing the coated drug pellets into tablets with an extra granular material.

12. A process for preparing a pharmaceutical composition of nebivolol having a specific surface area of less than 22×10³ cm²/g, by Extrusion-spheronization comprising:
(i) blending nebivolol with one or more excipients to form a blended mixture,
(ii) granulating the blended mixture of step (i) with a binder solution to form wet mass,
(iii) extruding the wet mass of step (ii) followed by spheronizing using a spherodizer to provide spheroids, and
(iv) lubricating the spheroids and compressing the spheroids into tablets or filling the spheroids into capsules.
